(19) United States
(12) Patent Application Publication
(10) Pub. No.: US 2021/0330215 A1
(43) Pub. Date: Oct. 28, 2021

(54) FOOT DATA ACQUISITION

(71) Applicant: Hewlett-Packard Development Company, L.P., Spring, TX (US)

(72) Inventors: Matthew G. LOPEZ, San Diego, CA (US); William D. Meyer, San Diego, CA (US); Brian R. Jung, San Diego, CA (US)

(21) Appl. No.: 16/481,240

(22) PCT Filed: Mar. 29, 2018
(86) PCT No.: PCT/US18/25273

§ 371 Date: Jul. 26, 2019

(51) Int. Cl. A61B 5/107; A43D 1/02

(52) U.S. Cl. CPC A61B 5/1078; A43D 1/022

(57) ABSTRACT

A foot data acquisition apparatus may include an array of inductor-capacitor (LC) tanks, a flexible wall opposite the array, an inflatable chamber between the array and the flexible wall and an electrically conductive material above the tanks between a top surface of the tanks and a top surface of the flexible wall.

### BACKGROUND

Characteristics of feet are sometimes measured to gather data that may be utilized to identify corrective orthotics and to form customized footwear. Such data may also be utilized by the podiatrist community to diagnose and quantify injuries and diseases, such as osteoporosis, muscular atrophy and diabetes, that may impact the foot or that are symptomatic in the foot.

### BRIEF DESCRIPTION OF THE DRAWINGS

FIG. 1 is a sectional view schematically illustrating portions of an example foot data acquisition apparatus.

FIG. 2 is a sectional view schematically illustrating portions of an example foot data acquisition apparatus.

FIG. 3 is a top view schematically illustrating portions of the example foot data acquisition apparatus of FIG. 2.

FIG. 4 is a schematic diagram illustrating an example circuit forming an example LC tank of the example foot data acquisition apparatus of FIG. 2.

FIG. 5 is a top view schematically illustrating portions of the example foot data acquisition apparatus of FIG. 2.

FIG. 6 is a top view schematically illustrating portions of an alternative implementation of the foot data acquisition apparatus of FIG. 2.

FIG. 7 is a sectional view schematically illustrating portions of an example foot data acquisition apparatus in an at rest state.

FIG. 8 is a sectional view schematically illustrating portions of the example foot data acquisition apparatus of FIG. 7 undergoing deformation in response to force is exerted by a foot.

FIG. 9 is a schematic diagram illustrating an example connection of an example controller to an example LC tank layer of the example foot data acquisition apparatus of FIG. 7.

FIG. 10 is a flow diagram of an example foot data acquisition method.

FIG. 11 is a perspective view illustrating portions of an example foot data acquisition apparatus in section.

FIG. 12 is a side view schematically illustrating the acquisition of foot data by the example foot data acquisition apparatus of FIG. 11 during a heel strike portion of a stride.

FIG. 13 is a side view schematically illustrating the acquisition of foot data by the example foot data acquisition apparatus of FIG. 11 during a foot flat stage portion of the stride.

FIG. 14 is a side view schematically illustrating the acquisition of foot data by the example foot data acquisition apparatus of FIG. 11 during a mid stance stage portion of the stride.

FIG. 15 is a side view schematically illustrating the acquisition of foot data by the example foot data acquisition apparatus of FIG. 11 during a foot flat stage portion of the stride.

FIG. 16 is a side view schematically illustrating the acquisition of foot data by the example foot data acquisition apparatus of FIG. 11 during a toe off stage portion of the stride.

FIG. 17 is a side view schematically illustrating portions of an example foot data acquisition apparatus.

FIG. 18 is a flow diagram of an example foot data acquisition method.

FIG. 19 is a flow diagram of an example foot data acquisition method.

Throughout the drawings, identical reference numbers designate similar, but not necessarily identical, elements. The figures are not necessarily to scale, and the size of some parts may be exaggerated to more clearly illustrate the example shown. Moreover, the drawings provide examples and/or implementations consistent with the description; however, the description is not limited to the examples and/or implementations provided in the drawings.

### DETAILED DESCRIPTION OF EXAMPLES

Disclosed herein are examples of a foot data acquisition apparatus, methods and a non-transitory computer-readable medium that facilitate the acquisition of foot data. The disclosed apparatus, methods and computer-readable medium utilize an array of inductor-capacitor (LC) tanks in combination with an inflatable chamber to accurately acquire profile data regarding the profile of a foot or feet. In one implementation, foot profile information is acquired for both feet. In other implementations, foot profile information is acquired for one foot at a time.

The example foot data acquisition apparatus, methods and computer-readable medium may acquire such foot data in a dynamic fashion, obtaining data that indicates how a foot's profile changes in response to different applied pressures. For example, the inflatable chamber may be inflated to different pressures to simulate different pressures experienced by a foot, such as different pressures experienced by the foot during a walking, jogging or running stride. In some implementations, the example foot data acquisition apparatus facilitates the acquisition of foot data while the person's foot or feet are walking, jogging or running across portions of the foot data acquisition apparatus. Such dynamic profile measurements may facilitate improved corrective orthotics and customized footwear. Such data may also improve upon the diagnosis and quantification of injuries and diseases, such as osteoporosis, muscular atrophy and diabetes, that may impact the foot or that are symptomatic in the foot.

Disclosed herein is an example foot data acquisition apparatus that may include an array of inductor-capacitor (LC) tanks, a flexible wall opposite the array, an inflatable chamber between the array and the flexible wall and an electrically conductive material above the tanks between a top surface of the tanks and a top surface of the flexible wall.

Disclosed herein is an example foot data acquisition method that may include inflating an inflatable chamber sandwiched between a flexible wall and an array of inductor capacitor (LC) tanks, wherein an electrically conductive material resides between a top surface of the flexible wall and the array, receiving signals from the array as the flexible wall is being deformed by an overlying foot; and determining a profile of the foot based upon signals from the array.

In one implementation, the method may further include inflating the inflatable chamber to a first pressure, determining a first profile of the foot based upon signals from the array while the inflatable chamber is at the first pressure, inflating the inflatable chamber to a second pressure different than the first pressure and determining a second profile of the foot based upon signals from the array while the inflatable chambers at the second pressure.

In one implementation, the profile of the foot is determined based upon signals of the array while the inflatable chamber is at a first inflation pressure. In such an implementation, the method may further include inflating a second inflatable chamber sandwiched between a second flexible wall and a second array of inductor capacitor (LC) tanks, wherein a second electrically conductive material resides between a top surface of the second flexible wall and the second array. Concurrently with the receipt of signals from the array while the inflatable chamber is at the first inflation pressure, the method may further include receiving second signals from the second array while the second inflatable chamber is at a second inflation pressure different than the first inflation pressure and determining a second profile of the second foot based upon signals from the second array.

Disclosed herein is an example non-transitory computer-readable medium containing foot data acquisition instructions to direct a processing unit to concurrently receive signals from a first set of inductor capacitor (LC) tanks underlying a 1st foot and a second set of LC tanks underlying a 2nd foot while the 1st foot and the 2nd foot are deforming at least one flexible wall supported above the first set of LC tank and above the second set of LC tanks by at least one inflatable chamber. The instructions further direct the processor to determine a first profile of the second foot based on signals from the first set of LC tanks and determine a second profile of the second foot based on signals from the second set of LC tanks.

FIG. 1 schematically illustrates portions of an example foot data acquisition apparatus 20. Foot data acquisition apparatus 20 utilizes an array of inductor-capacitor (LC) tanks in combination with an inflatable chamber to accurately acquire profile data regarding the profile of a foot or feet. In one implementation, foot profile information is acquired for both feet. In other implementations, foot profile information is acquired for one foot at a time. Foot data acquisition apparatus 20 comprises LC tank array 24, flexible wall 28, inflatable chamber 32 an electrically conductive material 36.

LC tank array 24 comprises a layer of LC tanks arranged in a two-dimensional array. In one implementation, LC tank array 24 is formed upon a circuit board, such as a fiberglass circuit board, which embodies inductors and capacitors forming the individual LC tanks of array 24. Array 24 has a surface area larger than the dimensions of an individual foot to be measured. In one implementation, array 24 has a surface area larger than dimensions of two feet of the person such that both feet may be concurrently measured. For example, in one implementation, array 24 has a length of at least one meter and a width of at least one meter.

In one implementation, the individual LC tanks each have a length of 428 mm and a width of 48 mm with a center-two-center pitch of less than or equal to 5 mm. Each LC tank outputs a self-resonance frequency which varies in response to movement of the electrically conductive material 36 relative to the LC tank, wherein the frequency may be translated to a distance. Distance measurements taken from each of the individual LC tank of the array facilitate the generation of a profile of the foot being measured.

Flexible wall 28 comprises a wall or panel of a flexible material opposite LC tank array 24. In one implementation, flexible wall 28 is formed from a compressible material that is also deformable and stretchable. In one implementation, flexible wall 28 is sufficiently stretchable or deformable so as to envelop or wrap about at least 15 mm of sides of a foot resting upon flexible wall 28. Flexible wall 28 provides an upper surface upon which a person's foot or feet may rest. The weight of the foot or the way to the feet is sufficient to cause a flexible wall 28 to bend or flex in a direction towards LC tank array 24. Such flexing causes the electrically conductive material 36 to be moved towards LC tank array 24, altering the resonance frequency of the signals provided by the individual LC tank of array 24.

Inflatable chamber 32 comprises a volume formed by a bladder or other structure which is inflatable and extends between the LC tank array 24 and flexible wall 28. Inflatable chamber 32 spaces flexible wall 28 from LC tank array 24. In one implementation, inflatable chamber 32 is inflated with a liquid. In yet another implementation, inflatable chamber 32 is inflatable with a gas, such as air. In one implementation, inflatable chamber 32 is partially defined by flexible wall 28. In another implementation, flexible wall 28 overlies the bladder or membrane defining inflatable chamber 32. In one implementation, inflatable chamber 32 has a fixed volume. In another implementation, inflatable chamber 32 is stretchable so as to change in volume in response to presses exerted upon in chamber 32 by a foot or feet.

Electrically conductive material 36 comprises an electrically conductive material that is above the array 24 of LC tanks between a top surface 38 of such tanks of the array 24 and a top surface 40 of flexible wall 28. In one implementation, the electrically conductive material 36 is formed between inflatable bladder 38 and flexible wall 40. For example, electrically conductive material 36 may comprise a layer of electrically conductive material on an underside of flexible wall 40 or on a top side of the inflatable chamber 32. In another implementation, the electrically conductive material 36 may be formed within or integrated within flexible wall 28. Examples of electrically conductive material include, but are not limited to, copper and aluminum. In one implementation, the electrically conductive material 36 is in the form of a metal fabric, such as silver, copper or aluminum impregnated rubber fibers formed on the exterior of flexible wall 28 or embedded within flexible wall 28. In response to force is exerted on flexible wall 28 by foot or feet, the electrically conductive material 36 is moved relative to the array 24 of LC tanks. The spacing of the electrically conductive material with respect to the LC conductive tanks of the array 24 cause such tanks to exhibit different resonance frequencies, wherein the different resonance frequencies may be measured and translated to a distance separating flexible wall 28 and array 24. The varying distances beneath and about the foot exerting forces upon flexible wall 28 may be used to determine a profile of the foot exerting such pressures upon flexible wall 28.

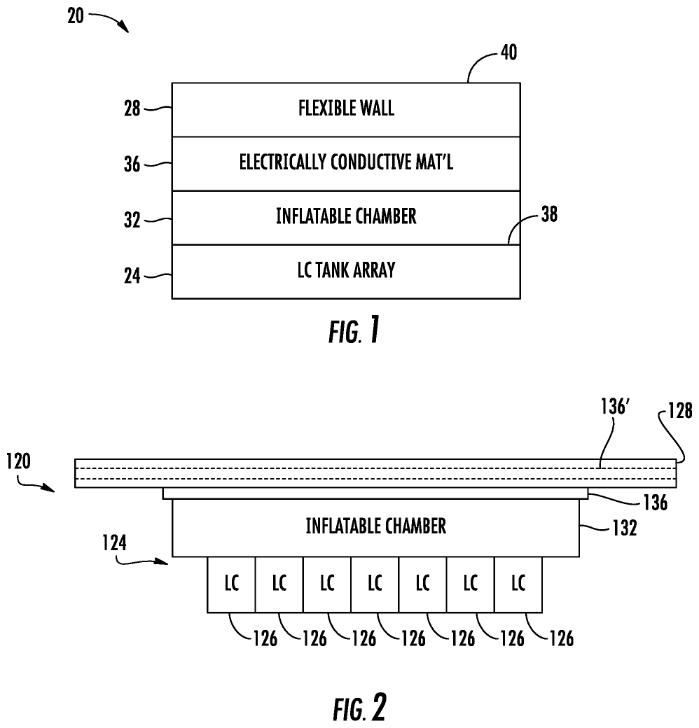

FIGS. 2 and 3 schematically illustrate portions of an example foot data acquisition apparatus 120. FIG. 2 is a sectional view while FIG. 3 is an enlarged top view schematically illustrating portions of apparatus 120. Apparatus 120 comprises an array 124 of individual LC tanks 126, flexible wall 128, inflatable chamber 132 an electrically conductive material 136. Array 124 of LC tanks 126 extends below inflatable chamber 132. In one implementation, array 124 is formed as part of a circuit board.

Array 124 has a resolution dependent upon the size of the individual LC tanks and the density of such LC tanks (number of LC tanks in a given area). Although array 124 is illustrated as being a 4×4 array of LC tanks in FIG. 3 for purposes of illustration, it should be appreciated that array 124 may have a much greater number of individual LC tanks 126. Array 24 has a surface area larger than the dimensions of an individual foot to be measured. In one implementation, array 24 has a surface area larger than dimensions of two feet of the person such that both feet may be concurrently measured. For example, in one implementation, array 24 has a length of at least one meter and a width of at least one meter. In one implementation, the individual LC tanks 126 each have an area less than the area of the overlying foot being measured. For example, in one implementation, the individual LC tanks 126 each have a length of 4 to 8 mm and a width of 4 to 8 mm with a center-to-center pitch of 4 to 8 mm. In one implementation, array 124 comprises an array of 512 by 512 LC tanks. In other implementations, array 124 may comprise other sized arrays.

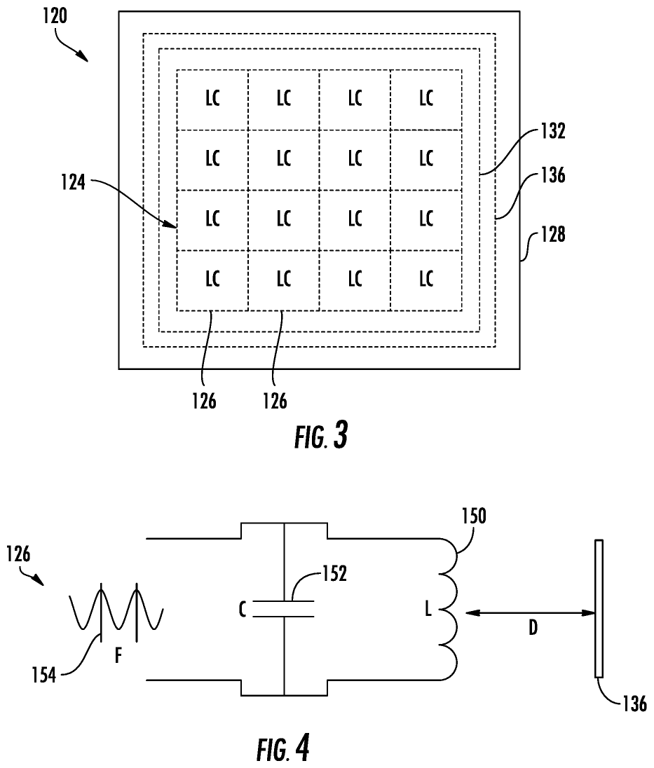

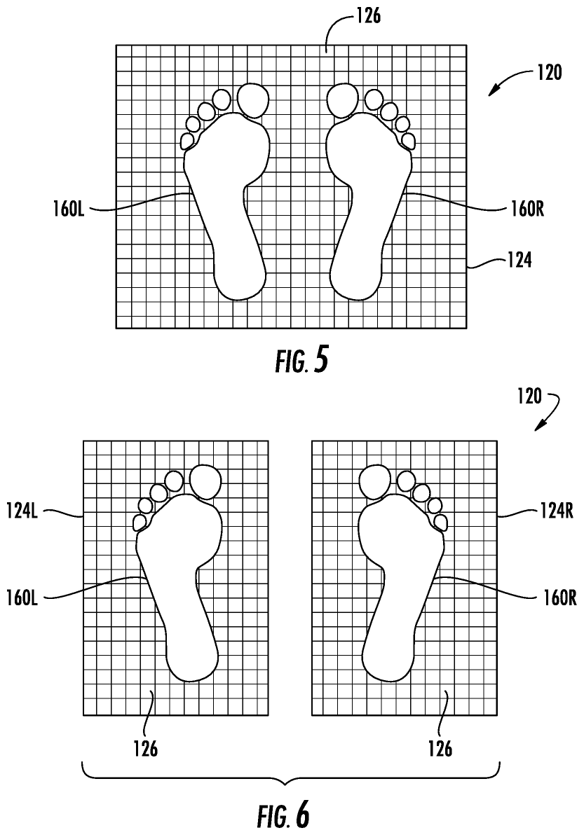

FIG. 4 schematically illustrates an example electrical circuit of one of LC tanks 126. LC tank 126 comprises an inductive coil 150 connected in parallel to a capacitor 152. Electrical current passing through the inductor 150 produces a magnetic field that interacts with the magnetic material 136 which results in the tank 126 resonating. Such resonance occurs at a frequency 154 which varies depending upon the distance D separating the magnetic material 136 and the inductive coil 150. In one implementation, the inductive coil comprises a multilevel coil connected to opposite sides of capacitor 152. The terminals of tank 126 output an electrical signal having a resonant frequency 154 (schematically shown) based upon a distance D separating the inductive coil 150 from magnetic material 136 (schematically shown). The resonant frequency 154 may be translated to a distance D. By determining the distance D for each of the LC tanks 126 of array 124, a profile of a foot may be determined.

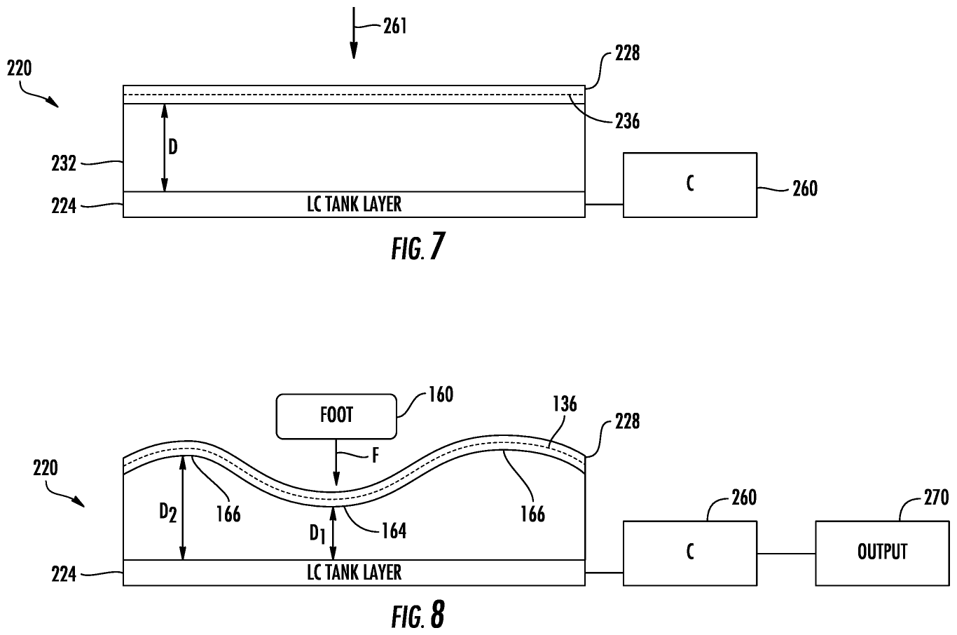

As shown by FIG. 5, in one implementation, array 124 comprises a single array of individual LC tanks 126 that covers a sufficiently large surface area such that both feet of a person may rest upon or over array 124 to facilitate concurrent profile measurements for both of feet 160L, 160R. As shown by FIG. 6, in another implementation, apparatus 120 may comprise two separate arrays 124L, 124R (collectively referred to as arrays 124), wherein each of arrays 124 has a sufficient surface area to underlie extend beyond a perimeter of the respective left and right feet 160L and 160R.

Flexible wall 128, inflatable chamber 132 and electrically conductive material 136 are similar to flexible wall 28, inflatable chamber 32 and electrically conductive material 36, respectively, as described above. In the example illustrated, inflatable chamber 132 extends outwardly beyond array 124. The electrically conductive material 136 extends outwardly beyond inflatable chamber 132. Flexible layer 128 extends outwardly beyond electrically conductive material 136. In other implementations, flexible wall 12A, inflatable chamber 132 and electrically conductive material 136 may be coextensive or may have other relative surface areas. In one implementation, a single flexible wall 128, a single inflatable chamber 132 in a single layer of electrically conductive material 136 may extend across an entirety of array 124. In yet other implementations, such structures may be provided by a plurality of such structures extending over the single array 124. For example, inflatable chamber 132 may comprise a plurality of inflatable compartments positioned adjacent one another. Likewise, flexible wall 128 and/or the layer of electric conductive material 136 may comprise a plurality of side-by-side members.

In those implementations where the foot data acquisition apparatus 120 comprises a separate array for each foot, such as arrays 124L and 124R, each of such arrays 124 may have a separate corresponding flexible wall 128, inflatable chamber 132 and layer of electrically conductive material 136. In some implementations, arrays 124L and 124R may share at least one of a flexible wall 128, inflatable chamber 132 and a single layer of electrically conductive material 136. Although layer of electrically conductive material 136 is illustrated as extending along an underside of flexible wall 128, in other implementations, the foot data acquisition apparatus 120 may comprise a layer of electrically conductive material 136′ formed within or embedded within flexible wall 128. For example, in one implementation, flexible wall 128 may include a layer of a metal fabric, such as a layer of silver, copper aluminum impregnated rubber material. In one implementation, the flexible wall formed from a polymer or rubber material which form dielectric layers about the electrically conductive material 136′. In some implementations, the layer of electrically conductive material forms flexible wall 128.

FIG. 7 schematically illustrates portions of an example foot data acquisition apparatus 220. Foot data acquisition apparatus 220 comprises LC tank layer 224, flexible wall 228 carrying an electrically conductive material 236 (shown in broken lines), inflatable chamber 232 and controller 260. LC tank layer 224 comprise a layer of LC tanks 126 (shown and described above) arranged in a two-dimensional array. As discussed above, each of the individual LC tanks 126 exhibit a resonant frequency that changes in response to changes in distance separating flexible layer 228 and layer 224.

Flexible wall 228 overlies LC tank layer 224. Flexible wall 228 is similar to flexible wall 28 or 128 described above. Flexible wall 228 changes shape in response to force is exerted upon flexible wall 228 in the direction indicated by arrow 261. In one implementation, flexible wall 228 is not stretchable and maintains a constant volume. In another implementation, such wall 228 is stretchable, changing in volume in response to forces exerted upon wall 228. In the example illustrated, flexible wall 228 as electrically conductive material 236 embedded therein. Electrically conductive material 236 causes changes in the resonant frequency as it moves closer to or farther away from the LC tanks 126 of LC tank layer 224.

Inflatable chamber 232 is similar to inflatable chamber 32 or 132 described above. Inflatable chamber 232 is sandwiched between flexible wall 228 and LC tank layer 224. In one implementation, flexible wall 228 may define inflatable chamber 232. In another implementation, flexible wall 228 may overlie the topmost wall of inflatable chamber 232. In one implementation, inflatable chamber 232 is filled with a liquid, such as water. In another implementation, inflatable chamber 232 is filled with a gas, such as air.

Controller 260 comprises a processing unit that follows instructions contained in a non-transitory computer-readable medium. Controller 260 is in communication with each of the LC tanks 126 of LC tank layer 224. In one implementation, controller 260 electrically stimulates each of the LC tanks 126 by sending individual pulses of electrical current. After stimulation of an individual LC tank 126, controller 260 receives electrical signals from the individual LC tank. In one implementation, controller 260 stimulates and/or receives electrical signals from the LC tanks 126 of LC tank layer 224 in parallel. In another implementation, controller 260 electrically stimulates and receives electrical signals from each of the individuals LC tanks 126 in series. In one implementation, controller 260 stimulates and receives signals from the individual LC tanks at a frequency of at least 200 Hz.

FIG. 8 illustrates the application of force F by foot 160 the top of flexible wall 228. As a result, flexible wall 228 changes in shape such that certain portions 164 of electrically conductive material 136 are moved closer to layer 224 while other portions 166 are moved further away from layer 224. This results in the different LC tanks 126 of layer 224 exhibiting different resonance frequencies. Controller 260 senses the different resonant frequencies and translates the different resonant frequencies to different distances such as the example distances D1, D2 shown. Controller 260 outputs foot profile data based upon the different determine distances to an output 270. The output 270 may be a display or may be a database or other memory storage. Such foot profile data may facilitate improved corrective orthotics and customized footwear. Such data may also improve upon the diagnosis and quantification of injuries and diseases, such as osteoporosis, muscular atrophy and diabetes, that may impact the foot or that are symptomatic in the foot.

FIG. 9 is a schematic diagram illustrating an example of how controller 260 may be connected to each of the individual LC tanks 126 of LC tank layer 224. As shown by FIG. 9, LC tank layer 24 is connected to a row multiplexer 272 and a column multiplexer 274. Each of the LC tanks 126 left and shown and described above) is connected to the row multiplexer 272 and the column multiplexer to 74. The row multiplexer 272 and the column multiplexer to 74 are each connected to controller 260 and a frequency digitizer 276. Controller 260 transmits electrical current to the LC tanks 126 through the row multiplexer 272 and the column multiplexer 274. The resulting resonant frequencies, dependent upon the individual distances of the individual LC tank 126 relative to flexible layer 228 and the magnetic material 136, is digitized by frequency digitizer 276 which transmits the digitized frequency values to controller 260. Controller 260 utilizes the digitized frequency values to determine the individual distances between the individual LC tanks 126 and individual opposing portions of layer 228. Using such information, controller 260 may generate an overall profile (shape and/or pressure) of the foot 160.

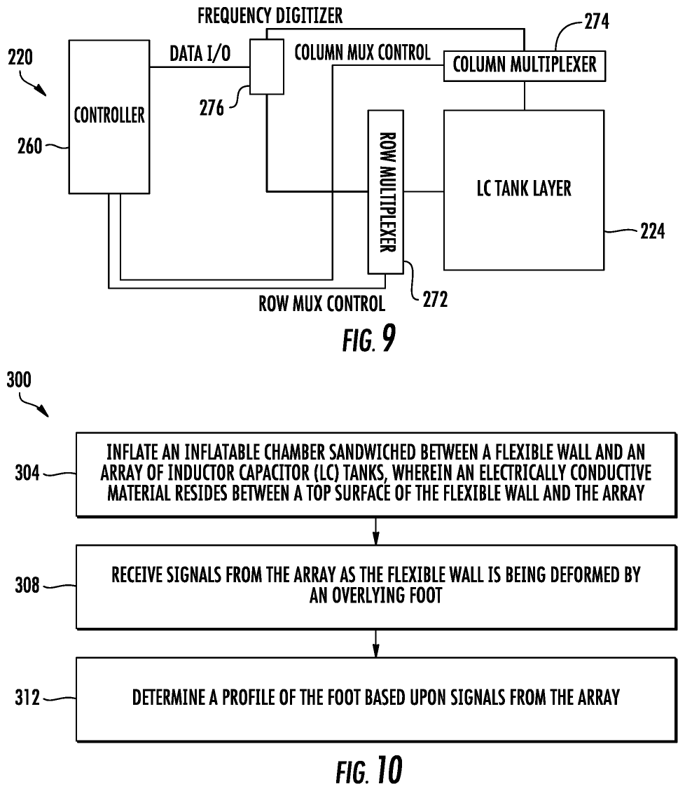

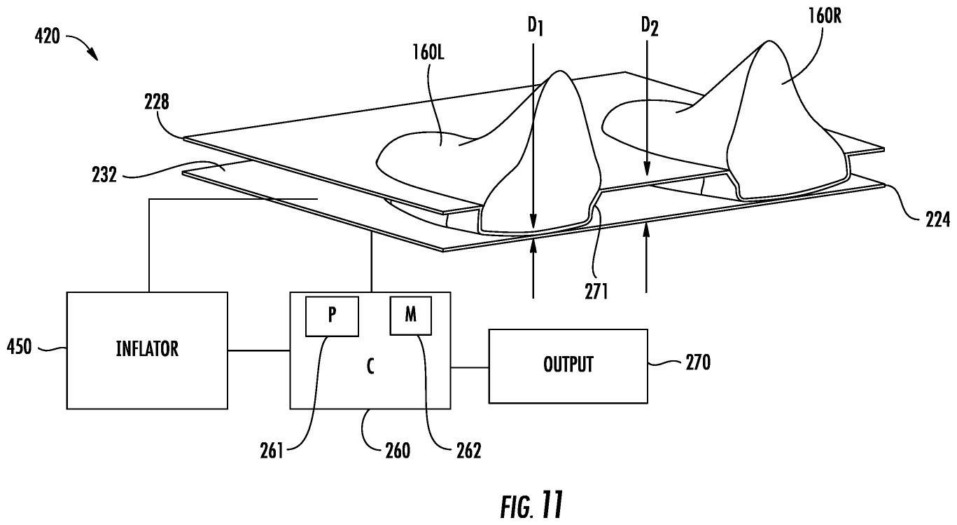

FIG. 10 is a flow diagram of an example foot data acquisition method 300. Method 300 utilizes an array of inductor-capacitor (LC) tanks in combination with an inflatable chamber to accurately acquire profile data regarding the profile of a foot or feet. Although method 300 is described in the context of being carried out by foot data acquisition apparatus 220, it should be appreciated that method 300 may likewise be carried out with any of the foot data acquisition apparatus described in this disclosure or similar apparatus.

As indicated by block 304, and inflatable chamber, such as chamber 232, sandwiched between a flexible wall, such as flexible wall 228, and an array of LC tanks, such as array 224) is inflated. An electrically conductive material resides between a top surface of the flexible wall in the array. As indicated by block 308, signals are received from the array as a flexible wall is being deformed by an overlying foot. The signals are a result of a resonant frequency of each of the LC tanks and correspond to the distance between the individual LC tanks and the flexible wall as well moving the electrically conductive material. As indicated by block 312, based upon the signals from the array, controller 270 determines a profile of the foot.

FIG. 11 is a perspective view illustrating portions of an example foot data acquisition apparatus 420 in section. Apparatus 420 is illustrated as being in the process of concurrently obtaining profile measurement data from two feet 160L and 160R. Apparatus 420 is similar to apparatus 220 described above except that apparatus 420 is specifically illustrated as additionally comprising inflator 450. Those remaining components or elements of apparatus 420 which correspond apparatus 220 are numbered similarly.

Inflator 450 comprises a device to selectively inflate inflation chamber 23 to one of many selectable pressures. Inflator 450 may comprise a pump for controllably pumping a liquid or gas into inflation chamber 232. In one implementation, inflator 450 may additionally comprise at least one valve to retain inflation chamber 232 at a selected pressure and/or to release fluid from chamber 232 to lower the pressure. Inflator 450 operates under the control of controller 260.

Controller 260 comprise a processing unit 261 that follows instructions provided in a non-transitory computer-readable medium 262 the instructions direct the processing unit 261 to output control signals controlling the operation of inflator 450 as well as the LC tanks 126 (shown in FIGS. 3 and 4) of LC tank layer 224. The instructions provided in memory 262 may direct processor 261 of controller 260 to carry out method 300 are any of the other methods described in this present disclosure. The instructions contained in memory 26 to direct processor 261 to translate the digitized resonant frequency values received from the individual LC tanks of LC tank layer 224 to individual distance values. In one implementation, such translation is carried out using an empirically determined formula using a digitized resonant frequency value as an input. In another implementation, such translation may be carried out by correlating the individual digitized resonance frequency values to individual distances using an empirically populated lookup table stored in memory 262.

As shown by FIG. 11, in one implementation, flexible layer 228 has a sufficient level of flexibility and inflation chamber 232 is inflated to a pressure such that the anticipated range of forces exerted upon layer 228 by feet 160 causes flexible layer 228 to deform or change shape, enveloping the perimeter or side surfaces of feet 160. In one implementation, flexible wall 228 is sufficiently stretchable or deformable so as to envelop or wrap about at least 15 mm of sides of a foot resting upon flexible wall 228. In one implementation, flexible layer 228 (and material 136) collectively form a layer having a durometer of 20 to 30 Shore A. In some implementations, controller 260 may prompt a person using apparatus 420 to enter his or her height and weight, wherein controller 260 selects an inflation pressure for chamber 232 based upon such entered information. As a result of such deformation of flexible wall 228 at a selected inflation pressure of inflation chamber 232, the bottom of feet 160 are separated from layer 224 by a first distance D1 while those regions of layer 224 along the sides or about feet 160 are spaced from layer 224 by a second distance D2. The transition region 271 may have a ramping distance which corresponds to the sides of the feet. Such different distances cause a change in inductance in each of the individual LC tanks, causing such individual LC tanks to exhibit different resonance frequencies. Based upon instructions contained in memory 262, processor 261 translate such different resonant frequencies into distance values and determines the profile of each of feet 160 using such distance values.

In addition to determining a shape profile of each of feet 160, controller 260 also determines a pressure profile of each of feet 160. In other words, not only does controller 160 determine the general shape and dimensioning of each of feet 160, controller 260 further determines the different degrees of force or pressure being exerted by the individual smaller regions or points of the foot 160L, 160R upon the underlying flexible layer 228. For example, different portions of the heel of each of feet 160 may exert different forces upon layer 228. Different portions of the ball or sole of the foot may exert different forces upon layer 228. In one implementation, controller 260 utilizes such information to further determine an arch height and instep using empirically determined arch heights and their corresponding pressure profiles. This pressure profile may further facilitate improved corrective orthotics and customized footwear. Such pressure profile data may also improve upon the diagnosis and quantification of injuries and diseases, such as osteoporosis, muscular atrophy and diabetes, that may impact the foot or that are symptomatic in the foot.

In some implementations, controller 260 may output control signals causing inflator 450 to inflate inflation chamber 232 to different inflation pressures. Such inflation pressure changes may be carried out in a stepwise manner or in a gradual ramped manner. At such different inflation pressures, controller 260 may receive signals from each of the LC tanks of layer 224 and determine shape and/or pressure profiles of feet 160. As a result, controller 260 may determine changes in the shape of feet 160 or the pressure profile of feet 160 that occur in response to different degrees of underlying support, different inflation pressures. Such information may prove invaluable in developing footwear, orthotics and the like.

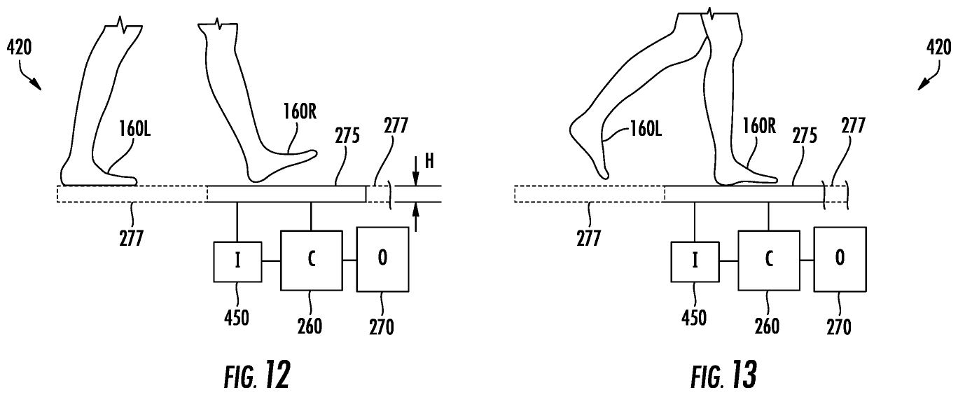

FIGS. 12-16 illustrate an example acquisition of foot data by apparatus 420. FIGS. 12-16 illustrate a person walking upon and over a sensing platform or pad 275 at least partially formed by layer 224, layer 228 (with electrically conductive material 136) and inflatable chamber 232 of FIG. 11. In one implementation, pad 275 has a thickness or height H that is less than or equal to 25 mm. As a result, pad 275 may be walked across as shown in FIGS. 12-16 without altering weight distribution characteristics during a stride. In one implementation, pad 275 the thickness or height H of less than or equal to 120 mm, further reducing any shifting of weight distribution characteristics during a stride that might otherwise occur as a result of a large degree of uneven or non-level support of the feet.

As shown by broken lines, in other implementations, pad 275 may have an enlarged area (additionally comprising region 277) sufficient to underline support both feet during a stride. For example, in one implementation, pad 275 may have a length of at least 1 m and a width of at least 1 m. In one implementation, region 277 also comprises layer 224, flexible layer 228 and inflatable chamber 232 such that the overall sensing area of pad 275 is sufficiently large to facilitate the concurrent acquisition of foot data from both of feet 160 during the illustrated stride. In another implementation, the sensing area of pad 275 may be limited to what is shown in solid lines while the broken line region 277 of pad 275 does not perform sensing. In such an implementation, the non-sensing portion 277 of pad 275 may be disconnected from controller 260 or may omit at least one of layer 224, inflatable chamber 232 of flexible layer 228.

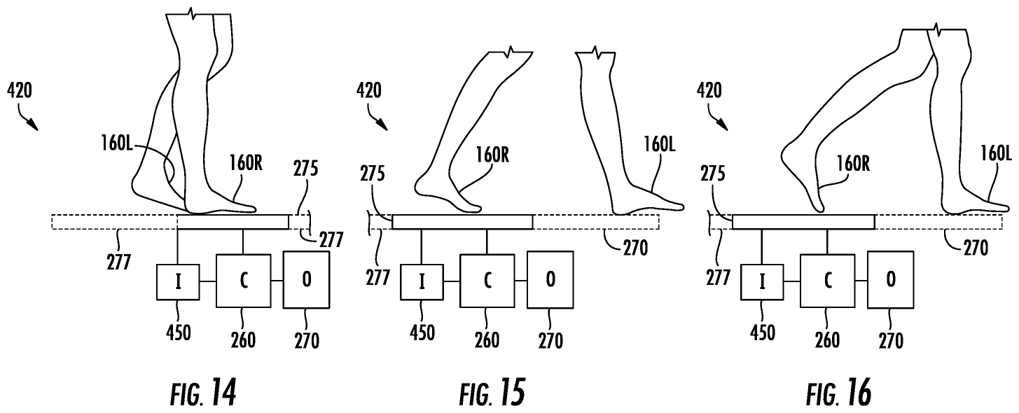

FIG. 12 illustrates foot 160R during a heel strike portion of a stride. FIG. 13 illustrates foot 160R during a foot flat portion of a stride. FIG. 14 illustrates foot 160R during a mid-stance. FIG. 15 illustrates foot 160R during a heel off portion of the stride. FIG. 16 illustrates the end of the stride, the toe off portion of the stride. During such portions of the illustrated stride, different underlying regions or portions of the foot 160R exert different pressures or forces upon pad. These pressures or forces vary from region to region of the foot. These pressures or forces also dynamically change from one stage of the stride to another stage of the stride.

During the stride, controller 260 outputs stimulus signals (electrical pulses) and receives the resulting resonant frequency signals (digitized or not digitized) at a frequency so as to dynamically determine foot shape or profile changes and foot pressure profile changes during each of the different stages or portions of the stride resulting from foot planting upon the flexible wall 228 of pad 275. In one implementation, controller 260 stimulation receives signals at a frequency of at least 200 Hz. As a result, controller 260 not only determines the shape and/or pressure for profile of the foot (or feet) in a static state, but also determines changes in the shape and/or pressure profile of the foot in response to changes in the force or pressure upon different portions of the foot as a person is walking. Similar measurements may be acquired during a jog or running, wherein the stride may be longer. In such implementations, the person may be prompted to jog or run across the platform or pad 275.

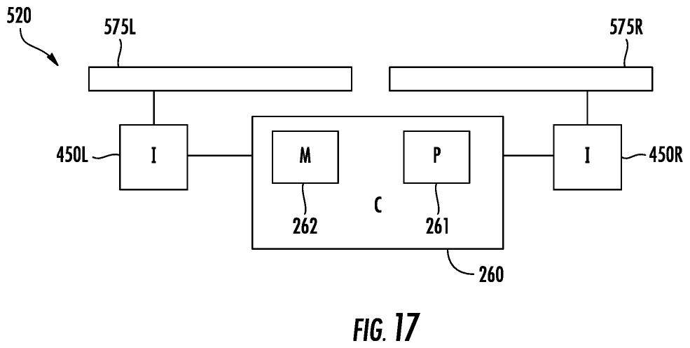

FIG. 17 schematically illustrates portions of an example foot data acquisition apparatus 520. Foot data acquisition apparatus 520 is similar to foot data acquisition apparatus 420 described above except that apparatus 520 comprises pads 575L and 575R (collectively referred to as pads 575), wherein each of pads is independently inflatable. Each of pads 575 is similar to pad 475 described above. Each of pads 575 comprises LC tank layer 224, flexible layer 228 (including electric conductive material 136) and inflatable chamber 232 described and illustrated above. In the example illustrated, pads 575L and 575R are associated with dedicated inflators 450L, 450R, respectively. In another implementation, a single inflator 450 may selectively and independently inflate the separate inflatable chambers 232 to different inflation pressures through the selective control at least one valve mechanism by controller 260. In some implementations, each of the inflatable chambers 232 (shown in FIG. 11) of pads 575 may have a pressure sensor which provides signals to controller 260 provide closed-loop feedback control over the operation of the at least one inflator 450.

The separate pads 575 having independent inflatable chambers 232 are inflatable to different pressures relative to one another. For example, the inflatable chamber 232 of pad 575L may be inflated to a first inflation pressure while the inflatable chamber 232 of pad 575R is inflated to a second inflation pressure different than the first inflation pressure. As a result, apparatus 520 may acquire foot data reflecting how different underlying pressure concurrently exerted upon each of the feet impacts and individuals foot shape and pressure profile. In some implementations, the inflation chambers 232 of the pads 575 may be alternated between different supporting inflation pressures so as to simulate the additional foot pressure forces encountered with walking, running or jogging.

FIG. 18 is a flow diagram of an example foot data acquisition method 600. Method 600 utilizes an array of inductor-capacitor (LC) tanks in combination with an inflatable chamber to accurately acquire profile data regarding the profile of a foot or feet. Although method 300 is described in the context of being carried out by foot data acquisition apparatus 520, it should be appreciated that method 600 may likewise be carried out with any of the foot data acquisition apparatus described in this disclosure or similar apparatus.

As indicated by block 604, controller 260 outputs control signals causing inflator 450R to inflate the inflatable chamber 232 of pad 575R to a first pressure. As indicated by block 608, while the inflatable chamber 232 of pad 575R is at the first pressure, controller 260 determines a first profile of the foot exerting force upon pad 575R. As indicated by block 612, controller 260 outputs control signals to inflator 450R to inflate the inflatable chamber 232 of pad 575R to a second pressure different than the first pressure. As indicated by block 616, while the inflatable chamber 232 of pad 575R is at the second pressure, controller 260 determines a second profile of the foot exerting force upon pad 575R. the first and second profiles may comprise a shape profile and/or a pressure profile of the foot exerting forces upon the pad 575R. In some implementations, method 600 may be concurrently carried out with respect to the other foot residing on the other pad 575R. In implementations where both feet are residing upon a single pad, such as one implementation of pad 275 described above, method 600 may also be concurrently carried out with respect to both feet. At such different inflation pressures, controller 260 may receive signals from each of the LC tanks of layer 224 and determine shape and/or pressure profiles of feet 160. As a result, controller 260 may determine how feet 160 respond or react to different degrees of underlying support, different inflation pressures.

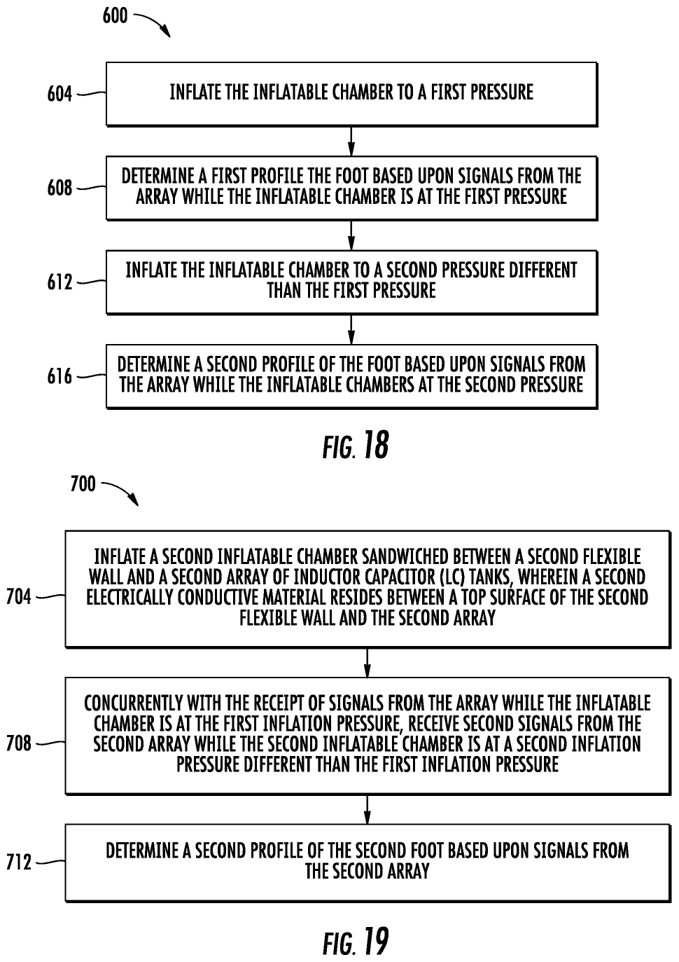

FIG. 19 is a flow diagram of an example foot data acquisition method 700. Method 700 utilizes an array of inductor-capacitor (LC) tanks in combination with an inflatable chamber to accurately acquire profile data regarding the profile of a foot or feet. Although method 700 is described in the context of being carried out by foot data acquisition apparatus 520, it should be appreciated that method 700 may likewise be carried out with any of the foot data acquisition apparatus described in this disclosure or similar apparatus.

Method 700 supplements method 300 described above with respect to FIG. 10. In other words, method 700 involves each of the actions described in block 304-312 as well as those described in block 704, 708 and 712. While the actions of blocks 304, 308 and 312 are carried out respect to pad 575L, the actions of blocks 704, 708 and 712 are carried out respect to pad 575R.

As indicated by block 704, controller 260 outputs control signals causing inflator 450R to inflate the inflatable chamber 232 of pad 575R. As indicated in block 704, the inflatable chamber being inflated is sandwiched between a second flexible wall 228 of pad 575R and a second array of LC tanks provided by a second LC tank layer of pad 575R. Pad 575R comprises a second electric conductive material that resides between a top surface of the second flexible wall and the second array.

As indicated by block 708, concurrently with the receipt of signals from the array of LC tanks of pad 575L and while the inflatable chamber 232 of pad 575L is at a first inflation pressure, controller 260 receives second signals from the second array of LC tanks of pad 575R while the second inflatable chamber 232 of pad 575R is at a second inflation pressure that is different than the first inflation pressure. As indicated by block 712, controller 260 determines a second profile of the second foot exerting forces upon pad 575R based upon signals from the second array of LC tanks of pad 575R. as a result, method 700 facilitates the determination of foot profile data (shape and/or pressure) as pads 575 are at different inflation pressures. The application of different underlying inflation pressures to the different feet may simulate the additional foot pressure forces encountered with walking, jogging and/or running, facilitating the acquisition of foot profile data for such actions while the person remained stationary upon pad 575.

Although the present disclosure has been described with reference to example implementations, workers skilled in the art will recognize that changes may be made in form and detail without departing from the spirit and scope of the claimed subject matter. For example, although different example implementations may have been described as including features providing various benefits, it is contemplated that the described features may be interchanged with one another or alternatively be combined with one another in the described example implementations or in other alternative implementations. Because the technology of the present disclosure is relatively complex, not all changes in the technology are foreseeable. The present disclosure described with reference to the example implementations and set forth in the following claims is manifestly intended to be as broad as possible. For example, unless specifically otherwise noted, the claims reciting a single particular element also encompass a plurality of such particular elements. The terms “first”, “second”, “third” and so on in the claims merely distinguish different elements and, unless otherwise stated, are not to be specifically associated with a particular order or particular numbering of elements in the disclosure.

What is claimed is:

1. A foot data acquisition apparatus comprising:
an array of inductor-capacitor (LC) tanks;
a flexible wall opposite the array;
an inflatable chamber between the array and the flexible wall; and
an electrically conductive material above the tanks between a top surface of the tanks and a top surface of the flexible wall.

2. The foot data acquisition apparatus of claim 1, wherein the array of inductor/capacitor tanks has a width of at least 1 m.

3. The foot data acquisition apparatus of claim 1, wherein the flexible wall has an area sized to underlie a first foot and a second foot of a person.

4. The foot data acquisition apparatus of claim 1, wherein a top of the flexible wall is spaced from a bottom of the apparatus by no greater than 15 mm.

5. The foot data acquisition apparatus of claim 1, wherein the electrically conductive material is carried by the flexible wall.

6. The foot data acquisition apparatus of claim 1 further comprising:
an inflator fluidly coupled to an interior of the inflatable chamber; and
a controller to output control signals to the inflator causing the inflator to selectively inflate the inflation chamber to a first inflation pressure and a second inflation pressure, and to determine a profile of a person's foot resting upon the flexible wall at each of the first inflation pressure and the second inflation pressure based upon signals from the array.

7. The foot data acquisition apparatus of claim 1 further comprising:
an inflator fluidly coupled to an interior of the inflatable chamber; and
a controller to output control signals to the inflator controlling inflation of the inflatable chamber by the inflator, wherein the controller outputs the control signals based upon signals from the array.

8. The foot data acquisition apparatus of claim 1 further comprising:
a second array of inductor-capacitor tanks;
a second flexible wall opposite the second array;
a second inflatable chamber between the array and the flexible wall; and
a second electrically conductive material above the second tanks between a top surface of the second tanks and a top surface of the second flexible wall, wherein the flexible wall and the second flexible wall are spaced and sized to concurrently underlie a first foot and a second foot, respectively.

9. The foot data acquisition apparatus of claim 8 further comprising:
a first inflator fluidly coupled to an interior of the inflatable chamber;
a second inflator coupled to an interior of the second inflatable chamber; and
a controller to output control signals to first inflator and the second inflator controlling inflation of the inflatable chamber by the first inflator and inflation of the second inflatable chamber by the second inflator, wherein the controller outputs control signals to concurrently inflate the inflatable chamber and the second inflatable chamber two different inflation pressures.

10. The foot data acquisition apparatus of claim 1 further comprising a controller in communication with the array of inductor-capacitor (LC) tanks, wherein the controller is to receive signals from each of the LC tanks in parallel.

11. The foot data acquisition apparatus of claim 1 further comprising a controller, wherein the controller is to receive signals from the array of LC tanks at a frequency so as to dynamically determine profile changes of a foot during different stages of the foot planting upon the flexible wall.

12. A foot data acquisition method comprising:
inflating an inflatable chamber sandwiched between a flexible wall and an array of inductor capacitor (LC) tanks, wherein an electrically conductive material resides between a top surface of the flexible wall and the array;
receiving signals from the array as the flexible wall is being deformed by an overlying foot;
determining a profile of the foot based upon signals from the array.

13. The foot data acquisition method of claim 12 comprising:
inflating the inflatable chamber to a first pressure;
determining a first profile of the foot based upon signals from the array while the inflatable chamber is at the first pressure;
inflating the inflatable chamber to a second pressure different than the first pressure;
determining a second profile of the foot based upon signals from the array while the inflatable chambers at the second pressure.

14. The foot data acquisition method of claim 12, wherein the profile of the foot is determined based upon signals of the array while the inflatable chamber is at a first inflation pressure, the method further comprising:
inflating a second inflatable chamber sandwiched between a second flexible wall and a second array of inductor capacitor (LC) tanks, wherein a second electrically conductive material resides between a top surface of the second flexible wall and the second array;
concurrently with the receipt of signals from the array while the inflatable chamber is at the first inflation pressure, receiving second signals from the second array while the second inflatable chamber is at a second inflation pressure different than the first inflation pressure;
determining a second profile of the second foot based upon signals from the second array.

15. A non-transitory computer-readable medium containing foot data acquisition instructions to direct a processing unit to:
concurrently receive signals from a first set of inductor capacitor (LC) tanks underlying a 1^{st} foot and a second set of LC tanks underlying a 2^{nd} foot while the 1^{st} foot and the 2^{nd} foot are deforming at least one flexible wall supported above the first set of LC tank and the second set of LC tanks by at least one inflatable chamber;
determine a first profile of the second foot based on signals from the first set of LC tanks; and
determine a second profile of the second foot based on signals from the second set of LC tanks.
